(19) United States
(12) Patent Application Publication
(10) Pub. No.: US 2005/0112148 A1
(43) Pub. Date: May 26, 2005

(54) Habit based self-help method for smoking cessation

(76) Inventor: Victor Matthew Dmitruk, Plainwell, MI (US)

Correspondence Address: Victor M. Dmitruk, 393 M-89, Plainwell, MI, 49080, US

(21) Appl. No.: 10/719,766

(22) Filed: Nov. 21, 2003

(51) Int. Cl. A61K 9/00; A61B 5/00

(52) U.S. Cl. 424/400; 600/300

(57) ABSTRACT

A habit-based method for smoking cessation. Smoking is a broadly based learned habit, and addiction is irrelevant to cessation. No medications or substances are used, and the Method does not require strict abstinence from smoking. Smoking is a habit broadly conditioned to commonly frequented locations in smoker's environment. Smoker using Cessation Method restricts smoking to previously not frequented, novel locations. Smoking is prohibited elsewhere. Smoking habit is thus gradually extinguished in commonly visited everyday locations. When extinction is complete, smoker avoids the identified previously not frequented smoking locations, and cessation is accomplished. Smoker also exhaustively identifies disadvantages to smoking and advantages to quitting to maintain motivation for cessation. These motives are employed to overwhelm the smoking habit. When smoker smokes in selected novel locations, the only activity allowed is review of disadvantages to smoking. When not smoking, smoker reviews advantages to quitting as a reward for abstinence.

### CROSS-REFERENCE TO RELATED APPLICATIONS

Not Applicable

### FEDERALLY SPONSORED RESEARCH

Not Applicable

### SEQUENCE LISTING

Not Applicable

### BACKGROUND OF THE INVENTION—FIELD OF INVENTION

This invention describes a process or a method for use by cigarette smokers and users of other tobacco products who would like to quit. In this instance, said invention or method or process relates to cessation of smoking and tobacco use, but said method or process applies equally to other excessive and repetitive habits, such as nail biting, thumb sucking, overeating, alcohol abuse, and the like.

### BACKGROUND OF THE INVENTION

Smoking is the most significant preventable risk factor for morbidity and mortality in the United States today. Although millions of smokers have quit in the decades since the health risks associated with smoking have been elucidated and better understood, approximately 25% of the adult population of the United States still smokes. This represents almost 50 million people, half of whom will die of a smoking related illness. Of those who die because of smoking related illness, one-half, or 12.5 million smokers will die in middle age, between the ages of 35 and 65. Prior to death, these smokers, collectively, will account for staggering losses to our economy in terms of health care costs, absenteeism, and diminished productivity. A study by Zinn (2003), concluded that the social costs of smoking cigarettes are three times that of illicit drug use. In 1990 more than 430,000 people died of smoking related illness in the United States, accounting for 26% of all deaths among men, and 17% of deaths among women. Lung cancer has now replaced breast cancer as the leading cancer killer in women. Approximately one-third of all cancer related deaths are attributable to smoking. Almost 90% of lung and oral cancer deaths and more than half of all neck cancer deaths are the direct result of tobacco use. Smoking has also been implicated in cancers of the cervix, stomach, liver, pancreas, and kidney, and it is linked to leukemia.

Cigarette smoking is not only a danger to the active smoker. Others exposed to Environmental Tobacco Smoke (ETS) carry an enhanced risk of developing the same medical problems as active smokers. The American Academy of Pediatrics reported that 43% of children in the United States between the ages of 2 and 11 years are exposed regularly to ETS. Some of the consequences of such exposure are low birth weight, asthma, middle ear disease, pneumonia, chronic cough, and recurrent respiratory infection. Exposure to ETS in children interferes almost immediately with both the growth and functioning of the lungs. In both adults and teens, smoking is associated with depression, anxiety, attention deficit-hyperactivity disorder, and a variety of other psychiatric conditions.

Smoking and exposure to ETS in pregnant women correlates with low birth weight, long-term cognitive and behavior problems, and sudden infant death syndrome. Smoking and exposure to ETS also reduces milk production in mothers who nurse, decreases the milk's vitamin C content, and increases colicky pain and diarrhea in breast fed infants.

Research studies and surveys have shown that fully 70% to 80% of smokers would like to quit smoking, but only about 5% accomplish smoking cessation on their own. A variety of therapies have been developed to assist smokers in quitting, notably the Nicotine Replacement Therapies (NRTs) and the antidepressant medication Bupropion (i.e., Zyban and Wellbutrin). Claims made by the manufacturers of these treatments are that they will “double quit rates”. However, few of the ads for these products reveal actual quit rates; improvement is only minuscule, increasing from the 5% natural quit rate to 10% or so. Additionally, significant side effects can result from the use of these products, including seizures and death, and relapse rates are generally very high. Approximately 80% of those quitting are smoking again within one year, and Yudkin (2003) reported no increase in quit rates after eight years in smokers who stopped smoking by using the nicotine patch. That is, 95% of the subjects in his study were still smoking eight years after “successfully” using the patch.

Virtually all NRT and medical approaches to smoking cessation claim that the likelihood of quitting is increased if the medical products are used in conjunction with a program including counseling or behavioral support. Stated somewhat differently, what research in this area demonstrates is that smokers can spend between $350 and $1500 for a smoking cessation program using NRTs or medications, with a 90% likelihood that they will fail. Additionally, it may be that the effectiveness observed is the result of counseling and behavioral support, and not the smoking cessation products.

Although use of NRTs and medications characterizes most approaches to smoking cessation, other approaches have been suggested and patented. Most fall into categories of (1) NRTs or other products to alleviate the symptoms of withdrawal from nicotine, (2) medications, (3) hypnosis, (4) acupuncture, (5) response prevention, or (6) operant conditioning using reward and punishment. For example, U.S. Pat. No. 6,294,193 to Cody (2001) was granted for an herbal composition containing Plantago Major and Hypericum that was intended to diminish desire for nicotine. U.S. Pat. No. 6,280,761 to Santus (2001) introduced a nicotine lozenge to reduce craving, and U.S. Pat. Nos. 5,284,163 and 5,293,883 to Knudsen, et al. (1994) and to Edwards (1994), respectively were based on cylindrical non-pyrolitic cigarette substitutes that released nicotine in granular or liquid form into the mouth. U.S. Pat. No. 6,344,222 to Cherukuri, et al. (2002) was granted for a medicated chewing gum containing nicotine, and U.S. Pat. No. 6,224,897 to Reitberg (2001) described a patient controlled flexible dosing system of nicotine delivery utilizing transdermal nicotine patches.

U.S. Pat. Nos. 5,965,567 to Archer, et al. (1999), 6,197,827 to Cary (2001), and 6,342,496 to Jerussi, et al. (2002) were all granted for proposed use of antidepressant medications and other chemical compounds to treat nicotine addiction.

Response prevention techniques to aid smokers in quitting were described in U.S. Pat. No. 5,590,421 to Craner (1997), who suggested a glove with digits attached (i.e., sewn together) that would make it difficult to pick up, hold, or manipulate a cigarette. In U.S. Pat. No. 6,458,149 to Peters-Combs (2002), an adult pacifier is described that is intended to be inserted into the mouth and sucked on until an urge to smoke passes. U.S. Pat. No. 4,951,691 to Leary (1990) describes a similar device consisting of an oval disk to be worn on a string about the neck and sucked on as a substitute for smoking. Presumably, the sensations associated with sucking the disk were to serve as acceptable replacements to the sensations experienced when one smokes cigarettes.

Approaches based on operant conditioning, punishment, and aversion were described in U.S. Pat. Nos. 4,068,672 to Guerra (1978), 4,269,203 to Corbett (1981), and 5,308,245 to Constantino (1994). Guerra's device was a cylindrical apparatus or cigarette holder that would deliver an electric shock when placed to the smoker's lips. Corbett's approach involved an apparatus that would hold smoker's pack of cigarettes, injecting a foul smelling substance into each cigarette before it is smoked. Constantino suggested a resilient, manually graspable tactile device about the size of a pack of cigarettes, that was intended to be grasped and tightly held by the smoker until a painfull burning developed in smoker's forearm. The device was then to be transferred to the other hand, and the operation repeated. At an extreme, U.S. Pat. No. 6,233,488 to Hess (2001) suggested a method the inventor claimed was particularly well suited to the suppression of nicotine craving. His method involved electrical stimulation of the spinal cord, using one or more surgically implanted leads, each containing at least two conducting electrodes.

What all of these approaches has in common is that, for one reason or another, they do not lead to smoking cessation. They are ineffective. Volumes of research evidence suggest that NRTs and medications may lead to improvements that are statistically significant, but they are not clinically significant. The number of smokers who are helped to quit after significant expenditures for these products, ranging from $350 to $1500 for the suggested 12-week course of treatment, is impressively small. Additionally, studies have shown that quit rates equal to or exceeding those claimed using NRTs and medications can be obtained by employing behavioral techniques alone, without NRTs or medications, and by scheduled smoking. In the latter instance, smokers attempting to quit commit to smoking at fixed intervals of time, and the intervals between cigarettes are gradually increased until abstinence is attained.

In his testimony on smoking cessation before the Senate Labor and Human Resources Committee in 1998, John Eisenberg, M.D., Administrator of AHCPR (Agency for Health Care Policy and Research), admonished physicians to address the issue of smoking with their patients. He noted that

Every patient who tries to quit should be offered effective treatments including social support, simple advice on how to quit successfully, and pharmacotherapies that have been demonstrated to increase the likelihood that a smoker will quit successfully (such as nicotine replacement therapies, like the patch and the gum, and the new non-nicotine medicine, Zyban, (italics mine).

Thus, despite overwhelming evidence that NRTs and medications are not effective aids to smoking cessation, physicians were encouraged to continue to prescribe these substances to their patients. The situation apparently has not changed in the intervening five years since Dr. Eisenberg made this address. McEwen and West (2003) reported that 86% of physicians surveyed in his study believed that nicotine replacements and medications were effective aids to smoking cessation and 83% recommended or prescribed them to their patients. This finding suggests that many physicians probably continue to prescribe these ineffective treatments, perhaps for lack of safe and effective alternatives.

Other approaches fail because smokers are unlikely to inflict pain upon themselves in the form of electric shocks or surgical implants, and still others because they lack face validity and appear silly. Few smokers would venture into their workplace or their favorite tavern or restaurant, sucking on a pacifier or disk attached to a string around their neck, or wearing gloves with the digits sewn together to make it difficult to manipulate small objects.

The current invention proposed for patent solves all of the problems and shortcomings of the techniques described above, and it is unique in that it is based on the following premises:

1. Smoking is based on habit rather than addiction to nicotine, and treating addiction is irrelevant to smoking cessation. The smoker may be addicted to nicotine because he or she smokes, but they do not smoke because they are addicted to nicotine. They smoke because smoking is an example of behavioral excess. It is a very pervasive and broadly conditioned bad habit, not unlike chewing gum, biting one's fingernails, drinking tea, coffee, or soft drinks, and eating or drinking to excess. It would be predicted that NRTs and medications would be minimally effective if the present analysis is correct, and this seems to be the case. In fact, Niaura (2002), concluded “Without any behavioral help, one should expect very low quit rates, on the order of 5%, with the nicotine patch”, i.e., equivalent to natural quit rates observed in smokers quitting on their own.
2. 70% to 80% of smokers do not, in fact, want to quit smoking. They may report they want to quit when queried about a self-destructive habit that, with a 50% likelihood, will lead to their premature death. However, they are not, in reality, motivated to quit. If they were, the “natural” quit rate would be greater than the abysmal 5% commonly reported. Thus, a significant part of any plan for smoking cessation must include provisions or methodology to first enhance, and then maintain, the smoker's desire to quit. Additionally each smoker's reasons for quitting are personal and unique. Each smoker's motives for quitting must be explored, identified, and used as a deterrent to smoking.
3. Contrary to statements made by most smokers, smokers do not enjoy smoking. They enjoy all of the activities in which they engage while smoking (e.g., watching television, meeting with friends, bowling, reading, driving, etc.).
4. Smoking cessation is not difficult to attain if approached properly, and the widely held belief that quitting is exceedingly difficult does nothing more than (1) deter smokers from attempting to quit, and (2) provide the smoker with a convenient rationale for continuing to smoke.
5. Ability to smoke on occasion must be included in any successful smoking cessation program. Including the ability to have a cigarette in the cessation plan prevents smokers from ruminating excessively about smoking, and lapses do not then constitute a failure. Instead, smoking is central to success, and allows for a natural titrating of nicotine in the smoker's system. This minimizes discomfort from nicotine withdrawal symptoms naturally, and eliminates the need for substances to reduce said discomfort.

This invention approaches the problems of smoking and smoking cessation from the standpoint of well-documented theories of learning and motivation. While learning theory approaches to smoking have been attempted in the past, they have failed. They failed because the particular learning theory applied to the problem of smoking was the wrong theory. It was theorizing based on what psychologists call “Effect Theory” of learning, or learning based on rewards and punishments. It is assumed by these approaches that one smokes because doing so is pleasurable, and that punishing the response of smoking should lead to its elimination. This is incorrect.

Another theoretical approach to learning is based on “Contiguity Theory”. Contiguity theory asserts that all that is necessary for learning or conditioning (i.e., habit development) is that a response occurs consistently in the presence of a particular stimulus. Thus, Pavlov's dogs learned to salivate to the sound of a metronome because food was injected into their mouths in the presence of a ticking metronome. The food consistently elicited salivation, and salivation was conditioned to the ticking sound.

Smoking is a response that occurs consistently in a wide variety of situations, and the response of smoking becomes conditioned to all. It becomes a very pervasive conditioned or learned habit, and this is why smokers believe they are addicted and need a cigarette. Their discomfort is not because of nicotine addiction, but rather the steady pull of the stimuli to which smokers are exposed on a daily basis, to which the response of smoking has been conditioned. These stimuli elicit conditioned responses that are experienced subjectively by smokers as a craving for a cigarette or for nicotine.

Smoking is a habit that has gotten out of hand, much like many other habits that collectively can be referred to as “behavioral excess”. A behavioral excess is a relatively benign behavior that becomes problematic when it occurs excessively. Furthermore, it is a behavior that can occur excessively because it is socially acceptable and can be practiced in a wide variety of situations. That is, because it can occur in a wide range of situations, it can be conditioned to all of them, and can easily get out of hand. For example, a small child can develop a problem of thumb-sucking because thumb-sucking in children is accepted in our society. However, many adults also suck their thumbs, but the problem does not get out of hand because our society frowns on adults who freely suck their thumbs in public. Adults must suck their thumbs privately, and very discretely. As another example, research has shown that about 20% of fingernail biters also bite their toenails. Fingernail biting is socially acceptable and becomes a behavioral excess and a problem. Toenail biting is not socially acceptable, and does not get out of hand. It becomes conditioned to only a very narrow range of stimuli.

A third example is masturbation. As with smoking, one might argue that masturbation is pleasurable, and that the pleasurable sensations are immediate rather than remote. Why then do we not find adults who masturbate as indiscriminately as smokers smoke? It is because the habit is not acceptable in public, and is contained and restricted to a very few stimulus settings. Consequently, it does not become behavioral excess.

According to contiguity theories of learning, a response is eliminated when a subject, in this case a smoker, continues to be exposed to previously effective elicitors of smoking and does not smoke. Not smoking, or some constellation of competing healthy responses, will then be conditioned to the stimuli previously eliciting smoking behavior. Once this occurs, the response is said to be extinguished.

This is one more reason for the lack of success of other smoking cessation programs, as they invariably counsel smokers to avoid situations in which they are likely to smoke. Because the “triggers” for smoking are avoided, the strength of the smoking habit in those situations remains strong, leading to relapse once the smokers return to their usual habits and activities. The key to quitting is to continue to frequent all of those situations, but to do so without smoking.

Another problem characteristic of most failed smoking cessation programs is the requirement of establishing a firm quit date, after which the smoker goes “Cold Turkey” and will no longer smoke at all. This results in constant craving and rumination about smoking, and smokers will eventually lapse and succumb to smoking one or a few cigarettes. This leads to relapse, as smoking just one cigarette is perceived by smokers as failure, and they begin to smoke once again. For example, smoking as few as 3 or 4 cigarettes in the course of a 12-week program using NRT or medication will result in only a 4% or 5% likelihood of quitting (i.e., exactly the same as the natural rate of quitting when no therapies are used). Therefore, smoking, or having an occasional cigarette in the course of attempting to quit must be integrated into any plan to quit. The recommendation for establishing a firm quit date, in reality, is not made because it enhances a smoker's ability to quit. It is made for the protection of the manufacturers of nicotine replacement therapies, as using their products and smoking poses serious health risks to the smoker because of the possibility of nicotine overdose.

In the present invention, smoker may smoke a cigarette at any time, but must do so under the strict guidelines of the program. When smoker does so, smoker is one step closer to quitting and becoming an ex-smoker. Having a cigarette is not perceived as failure, and does not lead to continued smoking.

Support for the effectiveness of the proposed invention has been before us for years, but has gone unnoticed because of the overwhelming bias that leads us to assume that smoking is based upon, and supported by, addiction to nicotine. This bias, and the mythology about the relationship between smoking behavior and addiction, is maintained through mis-education of the public and the medical community by the pharmaceutical giants that manufacture NRTs, Zyban, and Wellbutrin, and profit handsomely by touting their effectiveness in “doubling quit rates”.

One source of support for the current smoking cessation process or method lies in the fact that societal evolution in relation to smoking has naturally and severely limited the number and variety of settings in which one can smoke. Smoking has become less acceptable and less convenient, it consequently is conditioned to fewer settings, and this makes it easier for smokers to quit. For example, a referendum was passed in Florida in the 2002 midterm elections, prohibiting smoking in restaurants and many other public areas. California has passed similar “clean air” legislation in relation to smoking, and leads the Nation in limiting the venues in which smokers can practice their habit. The noose of limiting situations in which smokers can smoke has been gradually tightening over the past two decades, and millions of smokers have quit as a result.

Other evidence is found in the significant success of scheduled smoking in attaining smoking cessation when compared to results from use of NRTs or medications. Studies of scheduled smoking have demonstrated quit rates of up to 20% without the use of NRTs or medication. Scheduled smoking allows the smoker to continue customary daily routines, and does not require the smoker to avoid situations that trigger smoking, or to select a firm quit date. Smoker can continue to visit these situations, but does so without smoking because smoker is on the clock. Smoker also knows, however, that a cigarette is available when the scheduled smoking interval expires. Therefore, there is less rumination or craving for a cigarette as smoking is not taboo, and having a scheduled cigarette is not deemed to be failure. It is part of the treatment and cure. Additionally, NRTs, medications, or other practices to alleviate nicotine withdrawal symptoms are not needed with the present invention or with scheduled smoking. As smoker continues to smoke a few cigarettes under the strict conditions specified by said invention or method or process, nicotine is naturally titrated and symptoms of withdrawal are minimized.

Another bit of evidence to support inventor's contention of smoking as conditioned habit is the fact that merely restricting smoking in the workplace has been shown to increase quit rates by 5%. Once again, workplace restrictions on smoking make smoking less convenient and mean that smoker continues to visit all areas of the workplace, but does so without smoking. This results in a natural process of extinction of the smoking habit that makes smoking cessation easier. And, once again, smoker does not have to go Cold Turkey and does not ruminate excessively about smoking. Workplace restrictions are, in many respects, similar to scheduled smoking.

It is likely that the same mechanism of restricting smoking and a gradual process of extinction of the smoking response accounts for quitting and reduction of smoking in all of the examples described. The present invention magnifies and intensifies this process, as well as smoker's personal and idiosyncratic motivations, to accomplish rapid smoking cessation without relapse.

### SUMMARY

The invention, a habit-based self-help approach to quitting the smoking of cigarettes and the use of other tobacco products, is based on isolating smoking or tobacco use to a specific and rarely visited location so the conditioned response of smoking is extinguished in all other settings. Smoking may become conditioned or habitual in the rarely visited location, but smoker is then instructed to avoid said rarely visited location, and quitting is accomplished. Additionally, smoker's personal and idiosyncratic motivations for quitting are explored and identified, and said personal and idiosyncratic motives are used as deterrents to smoking, prior to smoker's decision to smoke in said rarely visited location.

Numerous advantages accrue to smoker because of said invention. Cost is nominal, as said invention does not require use of medications or nicotine replacements. Nicotine is naturally titrated, as smoker smokes fewer and fewer cigarettes in said remote location, minimizing withdrawal symptoms of nicotine addiction. As medications and nicotine replacements are not used, smoker is not exposed to potentially serious side effects, which have included nicotine overdose, seizures, and death with other approaches. Other advantages accrue in the event of lapse or relapse. In this invention, there are no quit dates; smoker may smoke at any time under the strict conditions of said invention, and there are no “lapses” or failures. Having a cigarette is part of the cure. In the event of relapse, all smoker must do is reinstate the program, with no additional cost. With other programs, smoker would be required to reinvest $350 to $1500, with another 95% likelihood of failure. As the present invention is a process or method presented in written form, cost can be nominal.

### DRAWINGS/FIGURES

FIG. 1. Graphic Representation of Smoker's Stimulus Constellation Prior to Instituting Invention.

FIG. 2. Graphic Representation of Smoker's Stimulus Constellation When Instituting Invention.

FIG. 3. Graphic Representation of Smoker's Stimulus Constellation after Successfully Using Invention.

### DETAILED DESCRIPTION

FIG. 1 represents the stimulus world of the smoker prior to attempting to quit, and before instituting said invention. All +s represent places smoker will frequent on a day to day basis in which smoking is permitted. These places include smoker's home, car, workplace, homes of smoking friends and relatives, taverns and smoking sections of restaurants, etc. The response and habit of smoking is conditioned to all of these situations, and the response of smoking frequently occurs. Xs refer to situations and places frequented by smoker where smoking is prohibited. These include government building, stores, hospitals, professional offices, etc. Smoking is not conditioned to these settings as the response does not occur in the presence of these cues. 0s represent situations in which smoking is permitted, but where smoker rarely ventures. Such places would include sitting on the side of a highway, under a tree in the woods, or behind a storage shed. Smoking is not conditioned to these situations, as smoker rarely ventures there, but they are critically important to said invention as it is in these situations that smoker will do all smoking when using said invention.

FIG. 2 represents the stimulus world of the smoker when utilizing said invention to quit smoking. All +s have now become Xs, and smoking is prohibited in all locations commonly frequented by smoker. This will lead to extinction of the habit in the presence of these cues. Smoking is now also prohibited in all but a very few 0s, even though smoker rarely visits these locations. The remaining 0s have become <>s. These are [image] [image] [image] locations that were previously visited very rarely, but are the locations in which smoker will smoke while utilizing said invention to quit. While the response of smoking is being extinguished in all locations that were previously sanctioned for smoking, it is being conditioned to a very small number of settings that previously were visited very rarely, if ever, by smoker. They have been selected under said invention for specifically this reason.

FIG. 3 represents the stimulus world of the non-smoker after successfully utilizing said invention to quit smoking. The response of smoking has been extinguished in the presence of all stimuli that previously elicited the habit, and smoking is prohibited in the presence of all Xs and 0s. The response of smoking is conditioned to all <>s, but ex-smoker is instructed to avoid these situations. Smoking cessation is now complete.

### OPERATION

The following steps are taken in implementing said invention for the purpose of smoking cessation, or the elimination of any similar behavioral excess.

(1) In step one, smoker keeps a smoking log for one week. Smoker identifies in the log (a) the time of each cigarette smoked, (b) the location or setting in which smoking occurred, (c) other parties present, and (d) the activity in which smoker was engaged. This identifies the +s, Xs, and 0s referred to in FIGS. 1-3.
(2) In step two, smoker develops two lists of personal motivations for quitting smoking. One list includes an exhaustive inventory of all of smoker's perceived disadvantages to continued smoking. These could be health related, financial, cosmetic, etc. The second list includes an exhaustive inventory of all of smoker's perceived advantages to quitting.
(3) In step three, smoker identifies a small number of <>s, or remote, infrequently visited locations in which all smoking will be done as smoker uses said invention to quit. Typically, one <> will be selected for use near smoker's home, another <> at smoker's workplace, and a third <> for traveling. For example, smoker may smoke behind a shed or garage at home, outside and away from others when at work, or stopping on the side of the road while traveling or commuting.
(4) In step four, smoker restricts all smoking to the designated <>s or smoking locations.
(5) In step five, smoker manipulates a Freedom Object, provided with said invention, whenever there is an urge to smoke. The Freedom is a small flat smooth stone or other similar object that smoker carries in pocket or purse. Smoker uses guided imagery in the form of all personal advantages to quitting while manipulating the Freedom Object to ward off the desire to smoke. Deep or controlled breathing and relaxation are also used to enhance smoker's positive imagery as a non-smoker.
(6) In step six, smoker deciding that smoking cannot be avoided goes to the <> selected for smoking and reviews all personal disadvantages to continuing to smoke. Smoker also commits in this step to doing nothing but smoking. No other party can be present, and all activities except smoking are prohibited. This is a test of smoker's enjoyment of smoking. Few people enjoy smoking in isolation, all the while thinking about illness, death and dying, and all of their other personal reasons for quitting.
(7) In step seven, smoker deciding not to smoke after implementing step six once again manipulates the Freedom Object and visualizes all of the advantages for quitting and a personal image of self as a non-smoker.
(8) In step 8, once smoker has made much progress in quitting, smoker is encouraged to avoid all <>s as smoker is now a non-smoker. Smoker will, however, continue to manipulate the Freedom Object and visualize the new found freedom and sense of personal well being and self-esteem that comes from being a non-smoker.
   These steps are repeated as needed each time the smoker has a desire for a cigarette, and are also repeated in the event of a lapse or a relapse.

1. What I claim as my invention is a self help method for eliminating smoking, tobacco use, and other similar repetitive habits:
a. which asserts smoking is based on habit and conditioned response and not addiction to nicotine, and
b. does not require the use of medications, nicotine replacements, or counseling, and
c. includes in said invention the opportunity for smoker to smoke so withdrawal symptoms are minimized and smoking does not constitute failure, and
d. which assumes smokers do not wish to quit and personal motives must be identified, magnified, and utilized to motivate smoker, and
e. which isolates smoking to unlikely locations so the habit can be extinguished or eliminated where it has commonly been practiced, and
f. which can be avoided once smoking cessation is accomplished, and
g. which utilizes positive and negative imagery in conjunction with relaxational techniques and a Freedom Object to enhance motivation to quit and to provide an alternative manipulandum for quitter to occupy quitter's hands when not smoking.
